(19) United States
(12) Patent Application Publication
(10) Pub. No.: US 2024/0426289 A1
(43) Pub. Date: Dec. 26, 2024

(54) MICROPUMP WITH INTEGRATED PIEZOELECTRIC TECHNOLOGIES FOR PROVIDING VALVE AND PUMP FUNCTIONALITY

(71) Applicant: AITA BIO INC., New York, NY (US)

(72) Inventors: Andrei Papou, San Jose, CA (US); Jacob Menture, New York, NY (US)

(21) Appl. No.: 18/274,135

(22) PCT Filed: Mar. 8, 2022
(86) PCT No.: PCT/US22/19395

§ 371 Date: Jul. 25, 2023

Related U.S. Application Data
(60) Provisional application No. 63/158,862, filed on Mar. 9, 2021.

(51) Int. Cl. F04B 43/04; A61M 5/142

(52) U.S. Cl. CPC F04B 43/046; A61M 5/142

(57) ABSTRACT

A MEMs micropump is disclosed that is configured as a substrate including a lower wafer and an upper wafer that together function as a pump and first and second valves on opposing sides of the pump, the micropump comprising: an inlet port for receiving fluid and an outlet port for releasing fluid, a chamber that communicates with the inlet and outlet ports, the chamber and inlet and outlet ports function together as a fluid path within the micropump; and a membrane, as part of the upper wafer, configured to deflect in and out of the fluid path, wherein the first and second valves each include a valve seat within the fluid path and a thin film piezoelectric layer configured to cause the membrane to deflect and engage the valve seat thereby preventing fluid flow within the fluid path; and wherein the pump including the chamber and a bulk piezoelectric layer for causing the membrane to deflect, thereby withdrawing fluid from the inlet port into the chamber and pumping fluid out of the chamber and outlet port.

### REFERENCE TO RELATED APPLICATIONS

This application claims priority to U.S. provisional application No. 63/158,862, filed on Mar. 9, 2021 entitled “Micropump With Integrated Piezoelectric Technologies For Providing Valve And Pump Functionality,” which is incorporated by reference herein.

### FIELD OF THE INVENTION

The present invention relates to a micropump with integrated piezoelectric technologies for providing valve and pump functionality.

### BACKGROUND OF THE INVENTION

Current pumping methods such as electric motor driven “plunger” pumps require high amounts of energy, are bulky, and are prone to mechanical failure. For several decades, the MEMS industry has attempted to build micropumps suitable for several applications such as fuel-cell injectors, gas-compressors, propulsive aerospace applications and underwater propulsive motion. In these MEMS micropumps, the actuators employed have ranged from electrostatic, electrochemical, thermoelectric, electromagnetic, and piezoelectric actuation (as examples). However, the medical device industry has failed to produce a commercially viable MEMs micropump for delivering medication. This is typically due to the inverse correlation between the flow rates required to deliver medication and the backpressure required to safely prevent unintended backward and forward flow over and or under delivery of the intended molecule.

### SUMMARY OF THE INVENTION

A micropump is disclosed with integrated piezoelectric (PZT) technologies for providing valve and pump functionality.

In accordance with an embodiment of the present disclosure, a MEMs micropump is disclosed that is configured as a substrate including a lower wafer and an upper wafer that together function as a pump and first and second valves on opposing sides of the pump, the micropump comprising: an inlet port for receiving fluid and an outlet port for releasing fluid, a chamber that communicates with the inlet and outlet ports, the chamber and inlet and outlet ports function together as a fluid path within the micropump; and a membrane, as part of the upper wafer, configured to deflect in and out of the fluid path, wherein the first and second valves each include a valve seat within the fluid path and a thin film piezoelectric layer configured to cause the membrane to deflect and engage the valve seat thereby preventing fluid flow within the fluid path; and wherein the pump including the chamber and a bulk piezoelectric layer for causing the membrane to deflect, thereby withdrawing fluid from the inlet port into the chamber and pumping fluid out of the chamber and outlet port.

In accordance with another embodiment of the disclosure, a micropump is disclosed having an architecture that is configured as a substrate including an inlet port for receiving fluid, outlet port for releasing the fluid and a chamber communicating with the inlet and outlet ports, the inlet port, outlet port and chamber forming a fluid path, the micropump comprising: a membrane configured to deflect in and out of the fluid path; a valve including a valve seat within the fluid path and a thin film piezoelectric layer configured to cause the membrane to deflect and engage the valve seat thereby preventing fluid flow within the fluid path; and a pump including the chamber and a bulk piezoelectric layer for causing the membrane to deflect, thereby withdrawing fluid from the inlet port into the chamber and pumping fluid out of the chamber and outlet port.

### BRIEF DESCRIPTION OF DRAWINGS

FIG. 1 depicts a cross sectional view of an example micropump within a device for delivering medication.

### DETAILED DESCRIPTION OF THE INVENTION

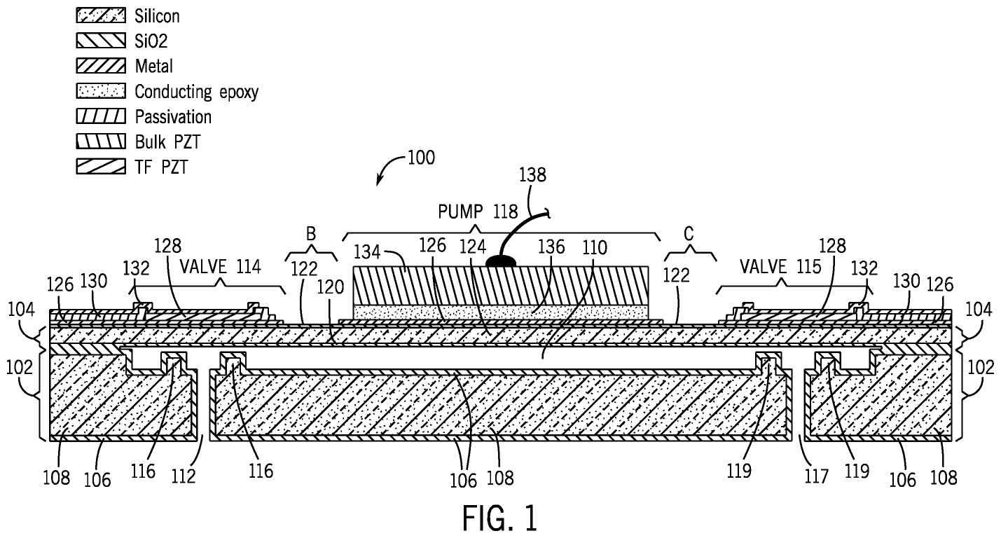

FIG. 1 depicts a cross sectional view of example micropump 100 or pumping unit within a device for delivering medication to a patient in an infusion system (not shown). The infusion system functions to infuse the medication or other fluid to a patient (i.e., a user of drug infusion system). The infusion system may be configured to infuse insulin, for example, to a patient for diabetes management (e.g., type 1). However, the infusion system can be configured to infuse other medications such as small molecule pharmaceutical solutions, large molecule or protein drug solutions, saline solutions, blood or other fluids known to those skilled in the art. However, micropump 100 may be used in other environments known to those skilled in the art.

In one configuration, the delivery device (or pod) described above is used to deliver insulin to a patient. The device includes micropump 100 (above) along with several other components such as a reservoir, a microcontroller unit (MCU) and an insulin delivery needle (not shown). The reservoir is configured to receive and store insulin for its delivery over a course of about three days, or as needed. However, reservoir size may be configured for storing any quantity of fluid as required. Micropump 100 fluidly communicates with reservoir to enable infusion as needed. In one configuration, an interposer may be used to connect the reservoir to micropump 100.

The delivery device may also include glucose monitoring components such as a continuous glucose monitoring (CGM), a sensor and needle (percutaneously inserted in the patient), a battery and a power controller (not shown). CGM, as known to those skilled in the art, tracks patient glucose levels and permits those levels to be used in algorithms that control flow rate. MCU controls the operation of micropump 100 to deliver insulin through the insulin needle from reservoir 14 at specific doses, i.e., flow rates over specified time intervals, based on CGM data converted to desired flow rate via control algorithms. The battery and power controller controls the power to the MCU and micropump 100 to enable those components to function properly as known to those skilled in the art. The CGM is powered by battery and the power controller through the MCU.

Micropump 100 incorporates a MEMS (micro-electro-mechanical systems) device, as known to those skilled in the art, that can be used for pumping fluid, valves used for regulating flow, actuators used for moving or controlling the pump and valves and sensor used for sensing pressure. In this embodiment, the MEMS device integrates different piezoelectric (PZT) technologies (or elements) as described in more detail below. Specifically, in the configuration described herein, micropump 100 integrates or includes both a bulk piezoelectric (PZT) as a pump actuator and a thin film piezoelectric (PZT) as a valve actuator in the same wafer and fabrication process flow, to reduce the size and ultimate cost of micropump 100 (as well as power reduction). In one example, micropump 100 incorporates a large pump chamber that is equipped with pick-and-place bulk ceramic PZT (or other piezoelectric material known to those skilled in the art) and a thin film PZT that is sputtered (or otherwise deposited) over a smaller active valve chambers using masking steps traditionally used for wafer processing. To this effect, micropump 100 built with a bulk (e.g., ceramic) PZT, as the pump actuator and thin film PZT, as valve actuators enables a reduction in micropump size and cost, while demonstrating sufficient flow rates required for medication delivery under back pressure conditions within micropump 100. That is, micropump 100 that employees both thin film PZT for the valve and bulk PZT for the pump as described herein can reliably deliver the necessary flow rates and backpressure tolerance to meet safety and reliability standards as well as reach size and form factor level needed for commercialization. (Micropump 100 may also be referred to as a reciprocating diaphragm/membrane pump or peristaltic pump.)

As indicated above, FIG. 1 depicts a cross-section view of micropump 100 as a substrate (or cavity substrate) that includes lower wafer 102 and upper wafer 104. Lower wafer 102 and upper wafer 104 function together as both a pump and two valves (i.e., inlet valve and outlet port for outlet valve), positioned on opposite sides of the pump. However, only inlet valve 114 is shown in FIG. 1 along with pump 118. Outlet valve 115 is shown on the opposing side of pump 118 in FIG. 1. This second valve is the same structurally as inlet valve 114 shown in FIG. 1. Much of the description herein applies to outlet valve 115 as well as described in more detail below.

Although lower wafer 102 and upper wafer 104 of micropump 100 in FIG. 1 are referenced herein, these wafers are shown together (along with additional layering) as a fully fabricated substrate or MEMS device subsequent to wafer fabrication processing, depicting the material layering structure resulting from such wafer fabrication processing. (Hence, the resulting substrate may also be referred to as a wafer.) However, the wafer fabrication processing is described briefly below along with material layering structure.

Lower wafer 102. As shown in FIG. 1, lower wafer 102 comprises silicon dioxide layer (SiO₂) 106 that is layered over silicon base layer 108 of the lower wafer 102, including cavities defining chamber or cavity 110 and inlet port 112. Inlet port 112 is the input of inlet valve 114 shown. To achieve this, as part of the microfabrication process, oxide is grown on silicon base layer 108 by thermal oxidation. (In this example, oxide thickness determines valve seat height, but in other examples, the thickness is not determinative). Then, silicon dioxide layer 106 is etched, stopping at silicon base layer 108 as specified. Then, lower wafer 102 is pattered and etched to create cavities including pumping chamber 110 and inlet via or port 112 (and outlet port 117) as well as valve seat 116 (and valve seat 119). (Note that inlet port 112, feeding inlet valve 114, outlet port 117 and chamber 110 form a fluid path as known to those skilled in the art.)

Upper wafer 104. Upper wafer 104 of micropump or substrate 100 includes silicon oxide layers 120, 122 on the top and bottom of silicon membrane layer 124. As for purpose of fabrication, silicon dioxide layer 120 is similarly created on silicon membrane layer 124, by thermal oxidation as described above, on the entire (bottom) side of upper wafer 104. Then, lower cavity wafer 102 and upper wafer 104 are bonded to form the substrate described above. A handle layer is then removed from silicon layer 124 (to remove bulk amount thereof). Thermal oxidation is then applied to create the silicon dioxide layer 122 on the entire top surface of upper wafer 104.

Metal layer 126 is then formed on silicon dioxide layer 122 as a first electrode layer for complete conduction along the entire layer surface. Metal layer 126 is patterned and then etched, stopping at the silicon dioxide layer 122 and leaving an opening A thereon. Thin film PZT layer 128 is then sputtered or otherwise blanket deposited on the wafer 104, patterned and etched as shown. In this configuration, thin film PZT layer 128 stops at metal layer 126 and on silicon dioxide layer 122. The thin film PZT is configured as a circular membrane or layer. However, other shapes may be used as known to those skilled in the art.

Passivation layer 130 is then deposited on wafer 104, patterned and etched to open and expose most of thin film PZT layer 128 for subsequent electrode layering formation. The etching stops at metal layer 126, at silicon dioxide layer 122 and at thin film PZT layer 128. This exposes an edge of metal layer 126. The passivation layer 130 is removed for proper layering of the pumping mechanism.

Metal layer 132 is then deposited on the wafer 104. Metal layer 130 functions as a second electrode of thin film PZT 128 for opposing the polarity of metal layer 126. Platinum (Pt) may be used as the electrode layers but those skilled in the art know that other materials may be used. Metal layer 132 is then patterned and etched, stopping at passivation layer 130, and exposing opening area B (and opening C). The remaining metal layer 132 is ultimately removed for proper fabrication and layering of the pumping mechanism. The ultimate fabrication thereby creates a valve or valving section of micropump 100 separate from the pump or pumping section of micropump 100 as described in more detail below.

As for the pumping section, bulk PZT layer 134 is added to the resulting substrate during assembly by way of conductive epoxy layer 136 that is deposited on exposed part of metal layer 126. (Both bulk PZT layer 134 and epoxy layer 136 leaving opening area B exposed.) As a result the valve section and pumping section are separate. Note that both the valve and pump sections incorporate a common membrane 124 in this micropump 100.

While not shown in FIG. 1, bulk PZT layer 134 is metalized on the top and bottom sides thereof to ensure even distribution of electric potential. A bond-wire 138 is attached to the top of metalized bulk PZT 134 as shown to induce voltage across bulk PZT layer and ultimately to deflect silicon membrane layer 124 underneath it.

In operation, voltage applied to metal layers 126, 132 causes the corresponding section of membrane layer 124 to deflect as a result of thin film piezoelectric (PZT) layer 128 (in either direction based on voltage). If silicon membrane layer 124 deflects inwardly, it will reach and seat against valve seats 116, 118, thereby preventing fluid flow within micropump 100. Voltage applied across the bond wire 138 (on bulk piezoelectric (PZT) layer 134) and metal layer 126 causes silicon membrane 124 to deflect, thereby pumping fluid out or drawing fluid in the chamber 110 as desired. In an example above, in the valve above, the silicon membrane of a thin film PZT may be 30-100 um. Bulk PZT technology may add 50-250 um of PZT ceramic. These are only examples. Other bulk PZT technologies may be employed to achieve desired results as known to those skilled in the art.

The construction described above is an example micropump 100. However, additional or less layering structure may be employed to achieve desired results. Other layering may be employed to achieve desired results.

By incorporating a bulk PZT (layer) as the actuator for a pump and a thin film PZT as the actuator for a valve in micropump 100 as described above, micropump 100 is able to demonstrate sufficient pumping performance to overcome backpressure conditions within micropump 100. (Backpressure may arise as a needle is inserted in a patient (user) or from outlet occlusion.) Specifically, thin film PZT, functioning as the valve (instead of a full bulk PZT for both valve and pump), enables a reduction in overall micropump size and power consumption relative to the range needed to achieve form-factor and commercial adoption. That is, a reduction in valve size translates in a reduction in overall micropump size. Reduction in size makes the micropump (and overall delivery device that houses it) smaller. A smaller delivery device translates into lower cost and less bulk. It may also translate into reduced power consumption.

In summary, the pump itself must be sufficient in size to deliver or deflect membrane adequately to pump fluid to overcome backpressure. So, the size of the pump section (dimensions), i.e., pump chamber and layering, must be maintained to achieve desired performance. The micropump design disclosed herein reduces the in-plane (XY) size (dimensions) of the valve while thinning down the membrane, to reduce the size of the micropump itself but also takes into consideration these factors in view of overall dimensions to ensure proper pumping functionality. While the size of the micropump has been reduced overall, the micropump disclosed herein ultimately increases the effective design area for the pump section itself, thus enabling higher stroke volume.

In more detail, a chip area occupied by a pump chamber is typically equal to the area occupied by either of two valves or slightly one larger valve. Reducing the chip size occupied by valves is limited by accompanied reduction in deflection. This ultimately increases hydraulic resistance of a valve in an open state. This in turn reduces operating frequency and, as a result, limits flow rate. Thus, valve size, membrane deflection and thickness (among others) are important considerations to ensure proper valve functionality. Micropump 100 does also take into account those considerations for proper pump functionality as described herein.

First order dimension considerations to achieve desired results described above may be explained by using a formula for displacement of circular membrane as follows:

$$w = C \frac{{PA}^{2}}{{Et}^{3}} ,$$

where w is displacement of the center of the membrane, C is a constant, P is pressure (distributed force), A is valve or pump membrane area, t is membrane thickness and E is Young's modulus (or modulus of elasticity) of membrane material, (i.e., the mechanical property that measures tensile stiffness of a solid material). In this respect, valve area optimization (i.e., valve size reduction) is limited by membrane displacement (maintaining it) which is compensated by a reduction of membrane thickness to maintain such membrane displacement. The cubic effect of membrane thickness can affect the membrane area (i.e., overcompensate quadratic effect). By using a thin film PZT, however, Young's modulus is increased (due to the Young's modulus of silicon compared to some other PZT materials). Thus, cubic effect of membrane thickness is balanced by the sum of quadratic effect of the membrane area and linear effect of material parameters. As a result, large valve deflection can be maintained with a smaller membrane using a thin film PZT for the valve of micropump 100 described herein.

Now, in order to compensate for any decrease in performance of the membrane of the pump under back pressure (due to loss in membrane thickness of the thin film PZT), micropump 100 also incorporates a bulk PZT as described above for the pump portion of micropump 100 to compensate for any loss in that membrane thickness. That is, the bulk PZT may be increased to compensate for decrease in membrane thickness. In this way, there is no need to reduce the pump membrane area to avoid any reduction in stroke volume and flow rate.

In sum, the parameters A, t, and E of the formula for displacement of circular membrane above are varied to reduce the valve size to achieve proper valve membrane displacement for flow considerations while maintaining pump functionality as known to those skilled in the art.

In summary, a horizontal reduction in the dimensions of the valve membrane (and hence overall size reduction of micropump 100) has several advantages. First, backpressure tolerance is improved through superior forward and backpressure tolerance. Second, operating voltage and current required are reduced for micropump operation to achieve the necessary deflections for valve opening and closing, allowing for the integration of the actively controllable and actuated valves needed. Third, cost savings may be achieved by greater utilization of the silicon wafer substrate and a more reliable bonding process in sputtering than other processes.

The configuration described herein involves two valves (one not shown in FIG. 1) and one pump. However, several valves and/or pumps may be used to achieve desired results as known to those skilled in the art. In addition, a single common membrane is shown for the valves and pump. However, a separate membranes may be employed for separate valves and pump that are assembled on an interposer for example.

It is to be understood that the disclosure teaches examples of the illustrative embodiments and that many variations of the invention can easily be devised by those skilled in the art after reading this disclosure and that the scope of the present invention is to be determined by the claims below.

1. A MEMs micropump configured as a substrate including a lower wafer and an upper wafer that together function as a pump and first and second valves on opposing sides of the pump, the micropump comprising:
an inlet port for receiving fluid and an outlet port for releasing fluid,
a chamber that communicates with the inlet and outlet ports, the chamber and inlet and outlet ports function together as a fluid path within the micropump; and
a membrane, as part of the upper wafer, configured to deflect in and out of the fluid path,
wherein the first and second valves each include a valve seat within the fluid path and a thin film piezoelectric layer configured to cause the membrane to deflect and engage the valve seat thereby preventing fluid flow within the fluid path; and
wherein the pump including the chamber and a bulk piezoelectric layer for causing the membrane to deflect, thereby withdrawing fluid from the inlet port into the chamber and pumping fluid out of the chamber and outlet port.

2. The MEMs micropump of claim 1 wherein the lower wafer comprises a silicon base layer and a silicon dioxide layer that is layered over the silicon base layer, wherein the bulk piezoelectric thickness is increased as thickness of the membrane is decreased.

3. The MEMs micropump of claim 1 wherein the membrane comprises a silicon layer and the upper wafer includes silicon dioxide layers on top and bottom of the membrane.

4. A micropump having an architecture that is configured as a substrate including an inlet port for receiving fluid, an outlet port for releasing the fluid and a chamber communicating with the inlet and outlet ports, the inlet port, outlet port and chamber forming a fluid path, the micropump comprising:
a membrane configured to deflect in and out of the fluid path;
a valve including a valve seat within the fluid path and a thin film piezoelectric layer configured to cause the membrane to deflect and engage the valve seat thereby preventing fluid flow within the fluid path; and
a pump including the chamber and a bulk piezoelectric layer for causing the membrane to deflect, thereby withdrawing fluid from the inlet port into the chamber and pumping fluid out of the chamber and outlet port.

5. The micropump of claim 1 wherein the bulk piezoelectric layer thickness is increased as thickness of the membrane is decreased.
